(19) United States
(12) Patent Application Publication
(10) Pub. No.: US 2022/0410512 A1
(43) Pub. Date: Dec. 29, 2022

(54) ENDOSCOPE, OPTICAL LAMINATE, AND MANUFACTURING METHOD FOR OPTICAL LAMINATE

(71) Applicant: OLYMPUS CORPORATION, Tokyo (JP)

(72) Inventor: Jumpei YONEYAMA, Minowa-machi, Kamiina-gun, Nagano (JP)

(73) Assignee: OLYMPUS CORPORATION, Tokyo (JP)

(21) Appl. No.: 17/901,080

(22) Filed: Sep. 1, 2022

Related U.S. Application Data
(63) Continuation of application No. PCT/JP2020/008667, filed on Mar. 2, 2020.

(51) Int. Cl. B29D 11/00; G02B 23/24; A61B 1/00

(52) U.S. Cl. CPC B29D 11/0073; G02B 23/243; A61B 1/00096; A61B 1/0011; B29D 11/00403; A61B 1/04

(57) ABSTRACT

An endoscope includes an optical laminate and an image sensor. The optical laminate includes a first optical member that is a glass lens where an optical window is formed in a planar substrate, including a recessed portion or a protruding portion around the optical window, and a second optical member having a flat surface facing the substrate of the first optical member, and including a protrusion made of resin for being fitted with the recessed portion or the protruding portion. The flat surface of the second optical member is a surface of a glass substrate on the first optical member side. A resin lens of the second optical member is arranged on a surface on an opposite side of the flat surface. A flat portion excluding the optical window and the recessed portion or the protruding portion is in contact with the flat surface of the second optical member.

### CROSS REFERENCE TO RELATED APPLICATION

This application is a continuation of International Patent Application No. PCT/JP2020/008667, having an international filing date of Mar. 2, 2020, which designated the United States, the entirety of which is incorporated herein by reference.

### BACKGROUND

A method of laminating a plurality of optical members to form an optical laminate has been conventionally known. The optical member mentioned herein is, for example, a lens, and the optical laminate is a lens unit in which a plurality of lenses is laminated.

When the plurality of lenses is laminated, alignment between the lenses is important. For example, the specification of U.S. Pat. No. 9,910,239 discloses a method of using a first optical element provided with a first alignment structure and a second optical element provided with a second alignment structure to perform alignment between the first optical element and the second optical element.

### SUMMARY

In accordance with one of some aspect, there is provided an endoscope comprising:

an optical laminate on which object light is incident, the object light being light from an object; and an image sensor that captures an image based on the object light that has passed through the optical laminate, wherein the optical laminate includes: a first optical member where an optical window is formed in a planar substrate, the first optical member including a recessed portion or a protruding portion around the optical window; and a second optical member that has a flat surface facing the substrate of the first optical member, and that includes, on the flat surface, a protrusion made of resin for being fitted with the recessed portion or the protruding portion, the first optical member is a glass lens, the second optical member includes a resin lens and a glass substrate, the flat surface of the second optical member is a surface of the glass substrate on the first optical member side, the resin lens is arranged on, out of surfaces of the glass substrate, a surface on an opposite side of the flat surface, and is not arranged on the flat surface, and a flat portion excluding the optical window and the recessed portion or the protruding portion among the substrate of the first optical member is in contact with the flat surface that is a substrate surface of the glass substrate of the second optical member.

In accordance with one of some aspect, there is provided an optical laminate comprising:

a first optical member where an optical window is formed in a planar substrate, the first optical member including a recessed portion or a protruding portion around the optical window; and a second optical member that has a flat surface facing the substrate of the first optical member, and that includes, on the flat surface, a protrusion made of resin for being fitted with the recessed portion or the protruding portion, wherein the first optical member is a glass lens, the second optical member includes a resin lens and a glass substrate, the flat surface of the second optical member is a surface of the glass substrate on the first optical member side, the resin lens is arranged on, out of surfaces of the glass substrate, a surface on an opposite side of the flat surface, and is not arranged on the flat surface, and a flat portion excluding the optical window and the recessed portion or the protruding portion among the substrate of the first optical member is in contact with the flat surface that is a substrate surface of the glass substrate of the second optical member.

In accordance with one of some aspect, there is provided a manufacturing method for an optical laminate, comprising: producing a glass lens by forming an optical window in a planar glass plate and forming a recessed portion or a protruding portion around the optical window of the planar glass plate; producing a lens unit including one or more lens wafers and having a flat surface; forming, on the flat surface of the lens unit, a protrusion made of resin; attaching a plurality of glass lenses to the lens unit by fitting the recessed portion or protruding portion of the glass lens with the protrusion of the lens unit; and performing singulation into a plurality of optical laminates by cutting a wafer, wherein the producing the glass lens includes execution of producing of the glass lens that has been subjected to singulation multiple times to produce a plurality of glass lenses, the attaching the plurality of glass lenses to the lens unit includes attaching the plurality of glass lenses to the lens unit by fitting the recessed portion or protruding portion of the glass lens with the protrusion of the lens unit and bringing a flat portion of the glass plate and the flat surface of the lens unit into contact with each other, the lens unit includes a resin lens and a glass substrate, the flat surface of the lens unit is a surface of the glass substrate on the glass lens side, and the producing the lens unit includes arranging the resin lens on an opposite side of the flat surface without arranging the resin lens on the flat surface.

### BRIEF DESCRIPTION OF THE DRAWINGS

FIG. 1 illustrates a configuration example of an optical laminate.

FIG. 2 is a perspective view illustrating an optical laminate before subjected to dicing.

FIG. 3 is a diagram for describing the flow of manufacturing the optical laminate.

FIG. 4 is a schematic diagram for describing a process of producing a glass lens by polishing.

FIGS. 5A and 5B are diagrams each illustrating a configuration of the glass lens.

FIGS. 6A to 6C are diagrams each illustrating a lamination process of laminating lens wafers.

FIGS. 7A and 7B are diagrams each illustrating a configuration of a protrusion.

FIG. 8A is a diagram for describing alignment and a bonding process. FIG. 8B is a diagram for describing a singulation process.

FIGS. 9A and 9B are diagrams for describing a configuration in which an adhesive is arranged aside from the protrusion.

FIG. 10A is a plan view of a first optical member. FIG. 10B is a plan view of a second optical member. FIG. 10C is a cross-section view of the optical laminate.

FIG. 11A is a plan view of the first optical member. FIG. 11B is a plan view of the second optical member. FIG. 11C is a cross-section view of the optical laminate.

FIG. 12A is a plan view of the first optical member. FIG. 12B is a plan view of the second optical member. FIG. 12C is a cross-section view of the optical laminate.

FIG. 13A is a plan view of the first optical member. FIG. 13B is a plan view of the second optical member.

FIGS. 14A and 14B are cross-section views of the optical laminate.

FIG. 15 is a plan view of the second optical member.

FIG. 16A illustrates a configuration example of an endoscope system including an endoscope. FIG. 16B illustrates a configuration example of an imaging module including the optical laminate and an image sensor.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Exemplary embodiments are described below. Note that the following exemplary embodiments do not in any way limit the scope of the content defined by the claims laid out herein. Note also that all of the elements described in the present embodiment should not necessarily be taken as essential elements.

### 1. Overview

First, a method in accordance with the present embodiment is described. As disclosed in the specification of U.S. Pat. No. 9,910,239 or the like, an optical laminate in which a plurality of optical members is laminated has been well known. The following description will be given of a lens unit in which a plurality of lenses is laminated. Combining the plurality of lenses can enhance optical performance in comparison with a case of using a single lens. Note that the optical member in accordance with the present embodiment is not limited to a member including a lens, and can be extended to another optical member.

When the plurality of lenses is laminated, alignment between the lenses is important. Specifically, a relative positional relationship between two lenses needs to be set so that an optical axis of a first lens and an optical axis of a second lens are matched with each other. The same applies to a case where there are three or more lenses.

In alignment between lenses, a method of arranging a structure for alignment in each lens is disclosed in the specification of U.S. Pat. No. 9,910,239 or the like. For example, in a case where one of lenses is fixed, the method is to search for a position at which structures for alignment are fitted with each other while moving the other of the lenses, and thereby perform alignment of the lenses. Thus, in assembling of the lens unit, there is a case where force is applied to the structure for alignment. In a case where the structure for alignment and the lens are integrally formed as described in the specification of U.S. Pat. No. 9,910,239, there is a case where optical performance decreases due to distortion of the lens itself. For example. the distortion of the lens causes a variation in height of an actual lens surface from an ideal curved surface of the lens.

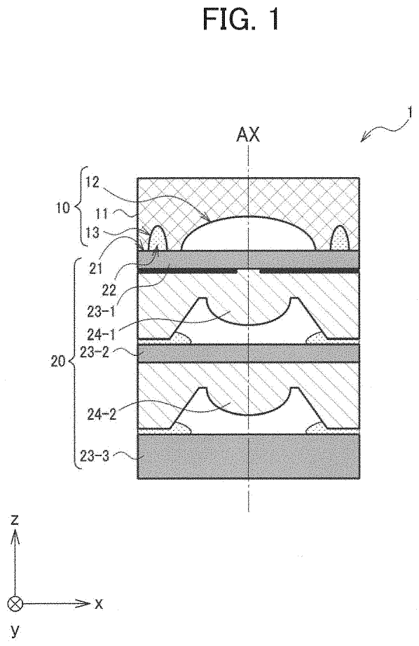

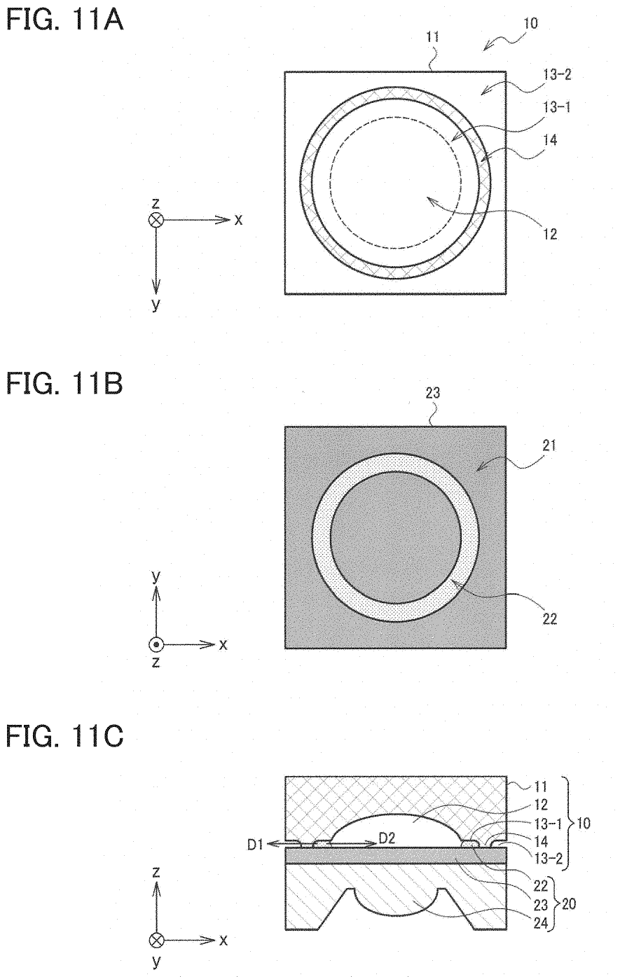

FIG. 1 is a cross-section view illustrating a configuration of an optical laminate 1 in accordance with the present embodiment. More specifically, FIG. 1 is a cross-section view of the optical laminate 1 on a plane including an optical axis AX. As illustrated in FIG. 1, the optical laminate 1 in accordance with the present embodiment includes: a first optical member 10 where an optical window 12 is formed in a planar substrate 11, the first optical member 10 including a recessed portion 13 around the optical window 12; and a second optical member that has a flat surface 21 facing the substrate 11 of the first optical member 10, and that includes, on the flat surface 21, a protrusion 22 made of resin for being fitted with the recessed portion 13. Note that as described later with reference to FIGS. 11A to 11C, the first optical member 10 may include a protruding portion 14 around the optical window 12. In this case, the protrusion 22 of the second optical member 20 is fitted with the protruding portion 14.

The optical window 12 of the first optical member 10 represents an incident port through which external light is incident on the optical laminate 1. The optical window 12 in accordance with the present embodiment may include a lens. In the first optical member 10, the optical window 12, and the recessed portion 13 are formed in the planar substrate 11. That is, the first optical member 10 is configured so that the structure for alignment and the lens are integrated with each other. Being around the optical window 12 represents a position surrounding the optical window 12 in a direction away from the optical window 12 with respect to the center of the optical window 12. More specifically, the recessed portion 13 is a groove that is arranged in a concentric pattern about the optical axis, as described later.

Meanwhile, the second optical member 20 includes the flat surface 21, and the protrusion 22 serving as the structure for alignment is formed on the flat surface 21. That is, the structure for alignment of the second optical member 20 is not formed integrally with the curved surface of the lens. For example, as illustrated in FIG. 1, the second optical member 20 may include a lens and a substrate. The flat surface 21 of the second optical member 20 is a surface of the substrate on the first optical member 10 side, and the lens is arranged on, out of surfaces of the substrate, a surface on the opposite side of the flat surface 21. The substrate mentioned herein is, for example, a glass substrate 23-1, and the lens is a resin lens (resin lens 24-1). In other words, in a case where the resin lens 24-1 is formed on a surface of the glass substrate 23-1 on one side, the flat surface 21 of the second optical member 20 is a surface of the glass substrate 23-1 on the other side. Note that the flat surface 21 mentioned herein is not limited to a perfect flat surface, and includes a substantially flat surface having minute irregularities. The minute irregularities are, specifically, irregularities whose heights or depths with respect to a reference plane are less than or equal to a threshold. Details of the other structures of the second optical member 20, such as glass substrates 23-2 and 23-3 and a resin lens 24-2, will be described later.

Even in a case where force is applied to the protrusion 22 serving as the structure for alignment of the second optical member 20, the method in accordance with the present embodiment prevents transmission of force to a portion that determines optical performance of the second optical member 20, specifically, the lens included in the second optical member 20. Hence, the method can prevent distortion of the lens due to alignment. At this time, usage of the glass substrate 23-1 that is less susceptible to deformation than the protrusion 22 further prevents transmission of force to the lens included in the second optical member 20. For example, even in a case where the resin lens 24-1 or the like that is susceptible to deformation is used as the lens included in the second optical member 20, optical performance is less likely to be decreased by alignment.

In the method in accordance with the present embodiment, resin is used for the structure for alignment on the second optical member 20 side. The resin has a characteristic of being susceptible to deformation in comparison with glass and the like. Hence, in a case where force is generated when the recessed portion 13 and the protrusion 22 are fitted with each other, deformation of the protrusion 22 prevents deformation of the first optical member 10.

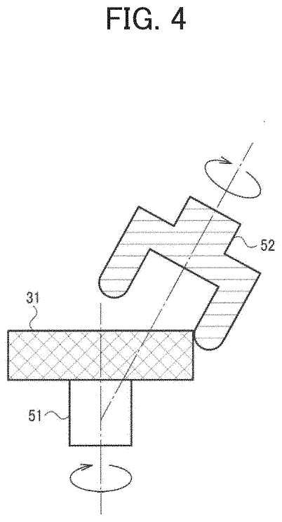

Subsequently, a difference between the present embodiment and the conventional method is described in terms of a material of an optical member. As a lens unit including a plurality of lenses, for example, a lens unit in which a plurality of glass lenses is laminated has been known. For example, the plurality of glass lenses is accommodated in a lens barrel to constitute the lens unit. Each glass lens included in the lens unit is produced by polishing, press molding using a mold, or the like, as described later with reference to FIG. 4.

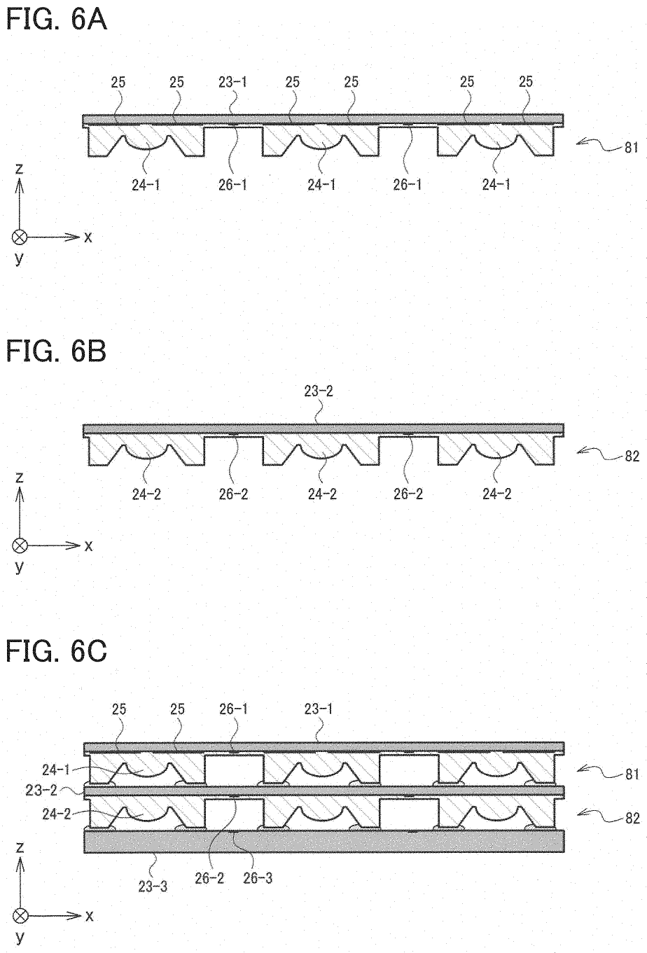

In addition, a method of using a semiconductor process to produce a lens unit serving as a wafer level laminate has also been known. For example, it is possible to collectively produce multitudes of lenses on a wafer by performing pattern transfer in which resin is sandwiched with a mold and cured with light or heat. Alternatively, multitudes of lenses may be produced using a step and repeat method. A wafer in which multitudes of lenses are formed on an identical plane of the wafer is hereinafter referred to as a lens wafer. A specific example of the lens wafer will be described later with reference to FIGS. 6A and 6B. Additionally, a method of laminating lens wafers to produce a laminated lens (wafer level lens) has also been known, as described later with reference to FIG. 6C.

In the lens unit using glass lenses, each lens unit needs to be manufactured individually. For example, the glass lenses are not formed collectively at a wafer level like resin lenses, but produced individually by polishing or the like. Thus, the lens unit in this case is produced by arrangement of a plurality of glass lenses that is individually produced at respective predetermined positions of the lens barrel. For this reason, it is difficult to increase productivity of the lens unit using the glass lenses, in comparison with a case of producing the lens wafer.

In contrast, it is difficult to increase a refractive index of the resin lens, which is widely used as a lens included in the lens wafer, in comparison with the glass lens. Hence, in a case where the lens unit with optical performance that satisfies a predetermined condition is produced by lamination of resin lenses, the number of laminated layers of lenses needs to be increased in comparison with the lens unit in which the glass lenses are laminated. As a result, the lens unit grows in size.

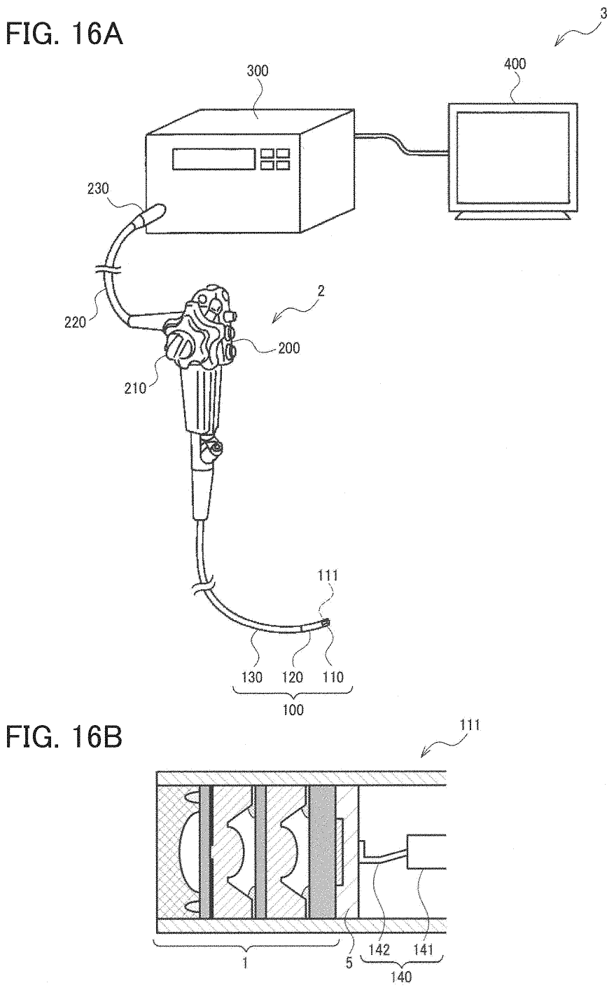

Especially, in a case where the lens unit is used for an optical system of an endoscope system 3, as described later with reference to FIG. 16A and FIG. 16B, the size of the lens unit has an effect on a configuration of an insertion section 100 of the endoscope system 3. In a case where the lens unit is large, for example, a rigid portion of a leading end section 110 becomes longer. As a result, there is a possibility that that flexibility of an operation becomes impaired. More specifically, it can be assumed that a bending operation of the leading end section 110 is not enough to change an imaging range, and an insertion and removal operation is needed.

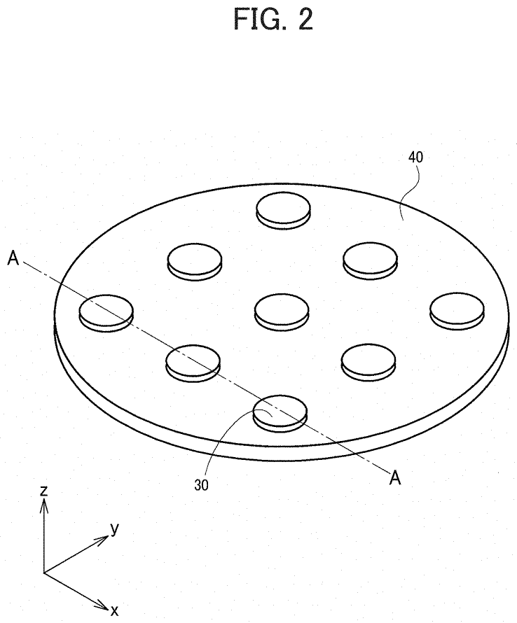

FIG. 2 is a perspective view for describing a process of manufacturing the optical laminate 1 in accordance with the present embodiment. For example, the lens unit in accordance with the present embodiment is produced by bonding of a plurality of glass lenses 30 in an aligned state to a laminated lens wafer 40 in which a plurality of lens wafers is laminated. Note that a target to which the glass lenses 30 are bonded is not limited to the laminated lens wafer 40, and may be a single-layer lens wafer. After the glass lenses 30 are bonded, the wafer is cut, and is thereby singulated into a plurality of optical laminates 1. The first optical member 10 in accordance with the present embodiment is, for example, the glass lens 30 illustrated in FIG. 2. The second optical member 20 in accordance with the present embodiment is, for example, part of the laminated lens wafer 40 illustrated in FIG. 2.

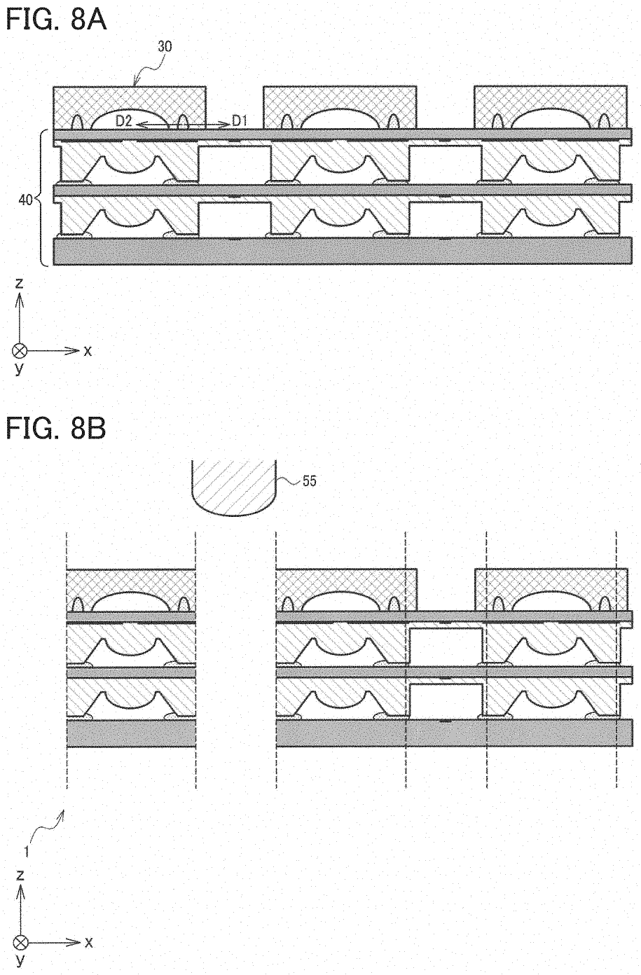

A coordinate system including an x-axis, y-axis, and a z-axis is defined below for the convenience of description. The coordinate system is, for example, a coordinate system defined using the laminated lens wafer 40 (second optical member 20) as a reference, and the z-axis is an axis that is parallel to the optical axis AX of the laminated lens wafer 40. The x-axis and the y-axis extend in a direction orthogonal to that of the z-axis, and an x-y plane is parallel to a wafer surface included in the laminated lens wafer 40. A cross-section view in the following description is, for example, a diagram illustrating a cross-section shape on a plane that is parallel to an x-z plane, and that includes an optical axis of the laminated lens wafer 40 or an optical axis of the second optical member 20 that is obtained by singulation of the laminated lens wafer 40. Especially, a section view before dicing is a diagram illustrating a cross-section shape on a plane that is parallel to the x-z plane, and that includes optical axes of a plurality of lenses. For example, FIG. 8B, which will be described later, illustrates a cross-section shape along A-A in FIG. 2.

In addition, the x-y plane is a plane that is parallel or substantially parallel to a flat surface of the substrate 11 of the first optical member 10, and the flat surface 21 of the second optical member 20. The first optical member 10 is laminated on the second optical member 20 in a direction along the z-axis. The direction in which the first optical member 10 and the second optical member 20 are laminated is also referred to as a laminating direction.

As described above, when the optical axis of the glass lens 30 is deviated from the optical axis of the laminated lens wafer 40, optical performance decreases. In this regard, the method in accordance with the present embodiment enables alignment between the first optical member 10 and the second optical member 20 in x and y directions with high accuracy.

In addition, the substrate 11 in accordance with the present embodiment has a planar shape. Thus, among the surface of the substrate 11 on the second optical member 20 side, at least part of a region other than the optical window 12 and the recessed portion 13 is a flat surface. More specifically, among the surface of the substrate 11 on the second optical member 20 side, the whole region other than the optical window 12 and the recessed portion 13 is the flat surface. That is, in the method in accordance with the present embodiment, the flat surface of since the substrate 11 and the flat surface 21 of the second optical member 20, which are both planar surfaces, are in contact with each other, it is expected to increase accuracy also in a z-direction. Especially, in a configuration without arranging an adhesive between the two planar surfaces, it is possible to increase accuracy in the z-direction.

Since it is possible to perform alignment with high accuracy in this manner, the method in accordance with the present embodiment enables matching of the optical axis of the glass lens 30 with the optical axis of the laminated lens wafer 40. Note that the matching mentioned herein is not limited to perfect matching and includes substantial matching with an error that is smaller than a given threshold. After the glass lenses 30 and the laminated lens wafer 40 are bonded to each other, the optical axis of each glass lens 30 or the first optical member 10, the optical axis of the laminated lens wafer 40 or the second optical member 20, and the optical axis AX of the whole of the optical laminate 1 are not distinguished from each other unless otherwise specified.

While FIG. 2 illustrates an example of bonding nine glass lenses 30 onto the laminated lens wafer 40, the number of optical laminates 1 that can be produced from one wafer can be modified in various manners.

Note that the first optical member 10 in accordance with the present embodiment is not limited to the glass lens 30 itself illustrated in FIG. 2. For example, part of the glass lens 30 may be cut when the wafer is cut. For example, removing a region outside an effective range as a lens, among the glass lens 30, at the time of dicing does not affect optical performance. In addition, a partial region of a bonding region may be removed at the time of dicing only if bonding strength can be secured. In this case, the first optical member 10 corresponds to the remaining portion after a peripheral portion is removed from the glass lens 30 illustrated in FIG. 2. A portion corresponding to the laminated lens wafer 40, among the optical laminate 1, is the second optical member 20.

The method in accordance with the present embodiment enables production of the second optical member 20 per wafer unit, and can thereby increase productivity in comparison with a case where the lens unit is manufactured by lamination of a plurality of glass lenses. In addition, since the first optical member 10 including a lens that has a relatively large refractive index can be added to the second optical member 20, it is possible to enhance optical performance in comparison with the laminated lens wafer 40 in which only the lens wafer including a resin lens is laminated. For example, it is possible to downsize the lens unit in comparison with a case where an attempt is made to implement equivalent optical performance by increasing the number of layers of the lens wafer.

When the laminated lens wafer 40 is manufactured, a mark for alignment used for a semiconductor manufacturing process can be conventionally utilized. The mark for alignment is, for example, an alignment mark 26(26-1 to 26-3), which will be described later with reference to FIGS. 6A to 6C. Since the lens wafer is formed and laminated using the alignment mark 26 as a reference, it is possible to perform alignment of the lens wafer having a plurality of layers with high accuracy. However, since the glass lens 30 is manufactured by metal molding or polishing, it is difficult to add the alignment mark. In a case of performing alignment of the glass lens 30 using the alignment mark 26 on the laminated lens wafer 40 as a reference, there is no choice but to align the outer shape of the glass lens 30 with the alignment mark 26. As a result, it is difficult to perform alignment between the optical axis of the glass lens 30 and the optical axis of the laminated lens wafer 40 with high accuracy, and this becomes a factor for a decrease in optical performance.

In contrast, the second optical member 20 in accordance with the present embodiment includes the protrusion 22 that has been aligned using the alignment mark 26. Since the alignment mark 26 is used for determination of the optical axis of the second optical member 20 as described above, the protrusion 22 arranged using the alignment mark 26 as the reference serves as a structure in which the position with respect to the optical axis of the laminated lens wafer 40 (second optical member 20) is set with high accuracy. The recessed portion 13 arranged in the glass lens 30 serves as a structure formed using the optical axis of the glass lens 30 as a reference. Using the protrusion 22 and the recessed portion 13 of the first optical member 10 increases accuracy in alignment regarding the optical axis, and can thereby enhance optical performance of the optical laminate 1.

### 2. Assembly of Optical Laminate

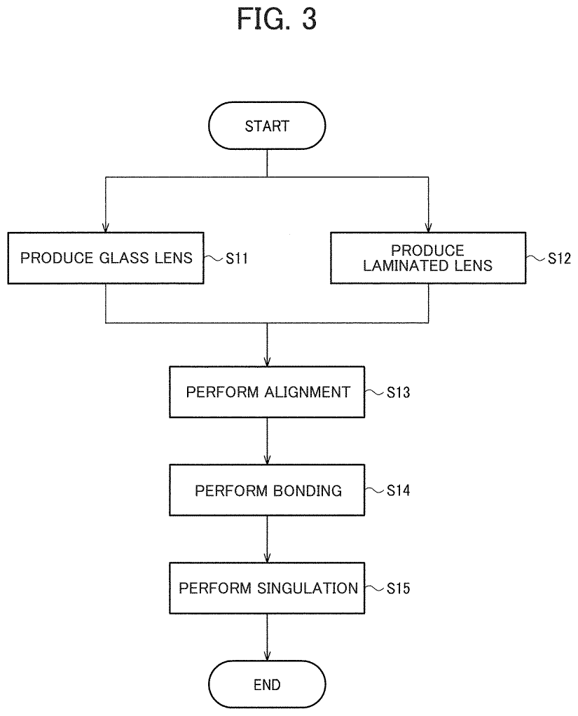

FIG. 3 is a diagram for describing the flow of assembling the optical laminate 1 in accordance with the present embodiment. In assembling of the optical laminate 1, first, the glass lens 30 is produced in step S11 and the laminated lens wafer 40 is produced in step S12. Note that either of the glass lens 30 and the laminated lens wafer 40 may be produced first, and both thereof may be produced in parallel. The production of the glass lens 30 described in step S11 includes production of the recessed portion 13. The production of the laminated lens wafer 40 described in step S12 includes production of the protrusion 22.

Subsequently, in step S13, alignment using the recessed portion 13 and the protrusion 22 is performed. After the alignment, in step S14, the glass lens 30 and the laminated lens wafer 40 are bonded to each other with an adhesive. In step S15, the glass lens 30 and the laminated lens wafer 40 that have been bonded to each other are singulated into a plurality of optical laminates 1 by dicing. Details of each step will be described below.

2.1 Production of Glass Lenses

The process of producing the glass lens 30 in step S11 is now described. The glass lens 30 may be manufactured by polishing or press molding using a metal mold.

FIG. 4 is a diagram for describing the process of producing the glass lens 30 by polishing. As illustrated in FIG. 4, a glass plate 31 is set on a rotating table 51. The glass plate 31 is ground with a grindstone 52 while the glass plate 31 is rotated using the rotating table 51, whereby the glass lens 30 is manufactured. Note that after the processes described in FIG. 4, processes similar to those performed in manufacturing of a normal glass lens, such as polishing with an abrasive, and centering and edging for aligning the optical axis and the center of a lens with each other may be performed.

As illustrated in FIG. 4, in manufacturing of the glass lens 30, a curved surface of the glass lens 30 is formed while the glass plate 31 is rotated. Thereafter, the recessed portion 13 is formed without removal of the glass plate 31 from the rotating table 51. With this process, the rotation center at the time of formation of the optical window 12 in the glass lens 30 and the rotation center at the time of formation of the recessed portion are common to each other. As a result, the recessed portion 13 can be formed at a position with the optical axis (optical center) of the glass lens serving as a reference. While the description herein has been given of the example of forming the recessed portion 13 after forming the optical window 12, the rotation center of the glass plate 31 is only required to be common in formation of each component, and the order of formation is not limited thereto. That is, these components may be simultaneously formed. The recessed portion 13 formed in this manner has a concentric pattern with respect to the optical axis. In other words, in a case where an attempt is made to arrange a structure with high positional accuracy with respect to the optical axis, it is difficult for the structure to have a pattern other than the concentric pattern.

Note that when press molding using a metal mold is performed, the metal mold is manufactured by rotation polishing. Hence, even in a case where the glass lens 30 is produced by the press molding, the point that the structure for alignment has the concentric pattern with respect to the optical axis is similar to the example illustrated in FIG. 4.

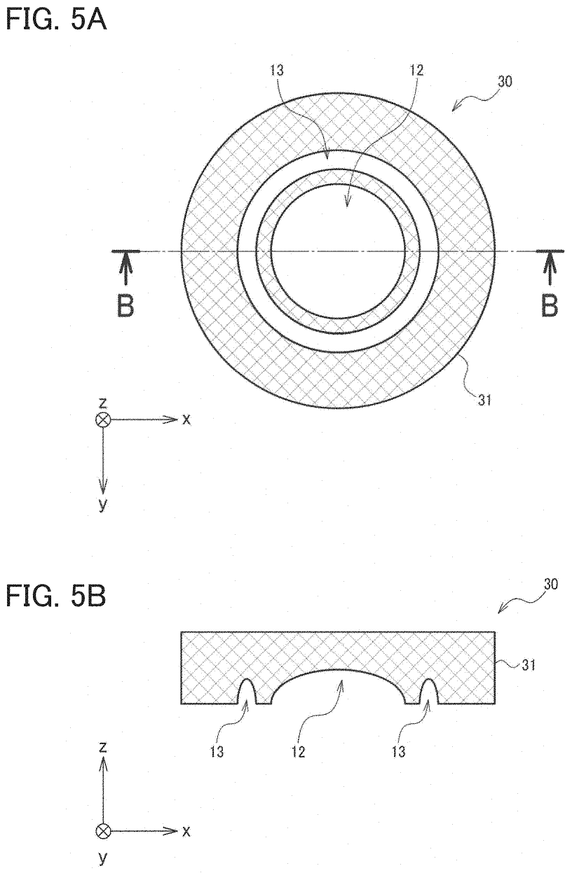

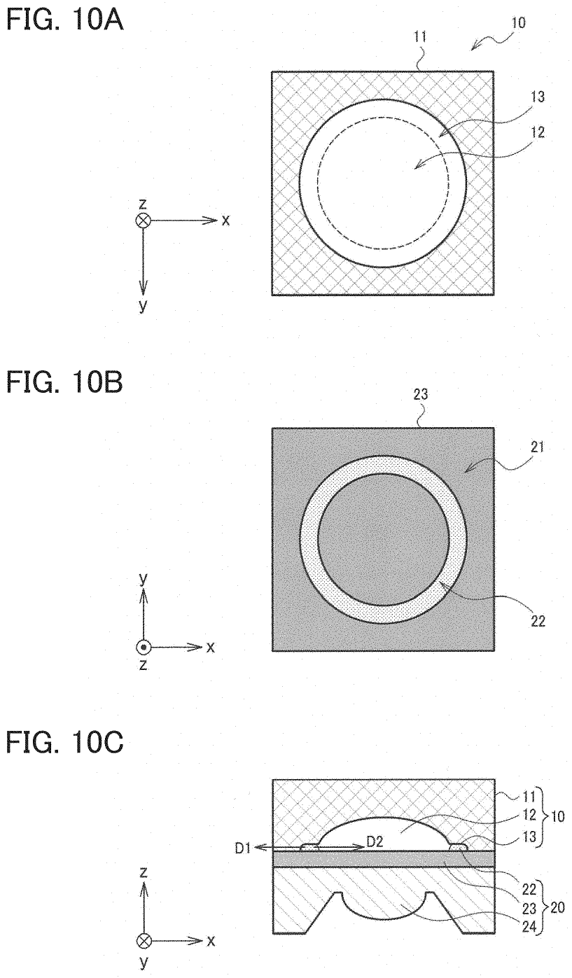

FIG. 5A is a plan view illustrating a configuration of the glass lens 30 in accordance with the present embodiment. The plan view mentioned herein represents a diagram when the glass lens 30 is observed from the surface thereof being in contact with the laminated lens wafer 40 in a state illustrated in FIG. 2. Note that the optical window 12 and the recessed portion 13, which are portions other than the flat surface, are not hatched for simplifying irregularities of the glass lens in FIG. 5A. This point is also applied to FIG. 10A and subsequent drawings. FIG. 5B is a cross-section view of a B-B plane of the first optical member 10. Note that each of x-, y-, and z-axes illustrated in FIGS. 5A and 5B represents a direction in a state where the glass lens 30 is bonded to the laminated lens wafer 40. While the description herein is given of the example in which the glass lens 30 is a disk, the shape of the glass lens 30 in a plan view can be freely determined. The plan view mentioned herein represents that a target object is observed from a point of view set at a position along the z-axis.

As illustrated in FIGS. 5A and 5B, the glass lens 30 is, for example, a planoconcave lens in which the optical window 12, which is a recessed portion having a circular shape in the plan view, is formed in a surface of the glass plate 31 on one side. Note that the glass lens 30 may be a lens having another configuration, such as a biconcave lens and a convex lens. The glass lens 30 includes the recessed portion 13, which is a concentric groove. As described above, the rotation center of the glass plate 31 at the time of formation of the optical window 12 and the rotation center at the time of formation of the recessed portion 13 are common to each other. Since the center of the optical window 12 corresponds to the center of a concentric circle corresponding to the recessed portions 13, the recessed portion 13 serves as a structure that is arranged using the optical axis of the glass lens 30 as a reference.

2.2 Production of Laminated Lens

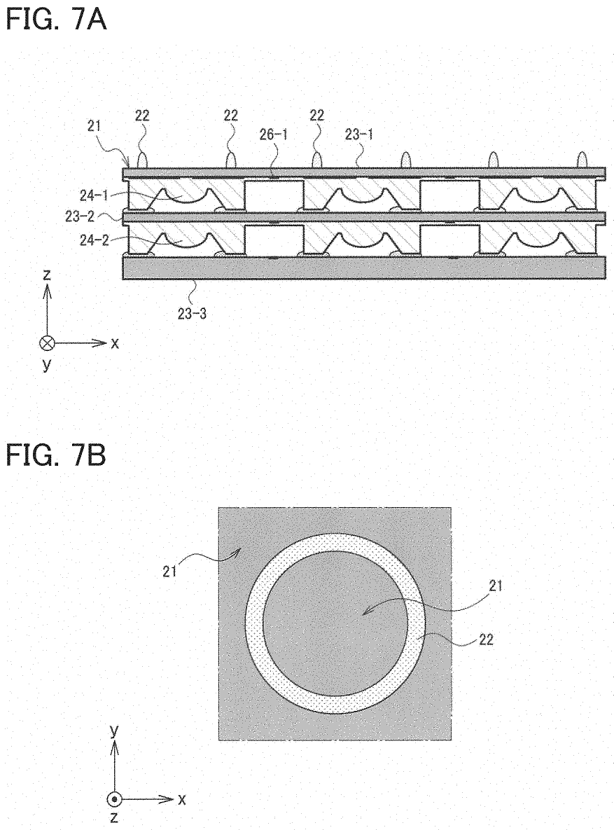

The process of producing the laminated lens wafer 40 in step S12 is now described. FIGS. 6A to 6C are diagrams for describing a lamination process of laminating lens wafers 81 and 82 included in the laminated lens wafer 40. FIGS. 7A and 7B are diagrams for describing a process for producing the protrusion 22.

Note that the x-, y-, z-axes illustrated in FIGS. 6C, 7A, and 7B correspond to respective axes illustrated in FIGS. 1 and 2. FIGS. 6A and 6B are cross-section views corresponding to part of FIGS. 6C and 7A, among the lens wafer before lamination.

FIG. 6A is a diagram illustrating a lens wafer 81 arranged at a position the closest to an object among the laminated lens wafer 40. An alignment mark 26-1 serving as a reference for alignment in the laminated lens wafer 40 and an aperture pattern 25 serving as an aperture are formed in a glass substrate 23-1. The alignment mark 26-1 and the aperture pattern 25 are formed using a patterning method for a semiconductor manufacturing process. Subsequently, a lens array including a plurality of resin lenses 24-1 is arranged at a position with the alignment mark 26-1 serving as references. The lens array is, for example, produced by subsequent execution of dropping of a photo-curable resin, press-fitting in a mold, and irradiation of light.

FIG. 6B is a diagram for describing a process of producing a lens wafer 82 that is relatively far from the object among the laminated lens wafer 40. The lens wafer 82 includes a glass substrate 23-2, an alignment mark 26-2, and a lens array including a plurality of resin lenses 24-2 formed on the glass substrate 23-2. The lens array is formed at a position with the alignment mark 26-2 serving as a reference. The lens wafer 82 is similar to the lens wafer 81 except a point that the aperture pattern 25 is not necessary.

As illustrated in FIG. 6C, the laminated lens wafer 40 is produced by lamination of the lens wafers 81 and 82. In the lamination process, the glass substrate 23-3 and the lens wafer 82 are bonded to each other, and the lens wafer 82 and the lens wafer 81 are bonded to each other. The bonding is performed using an adhesive. Note that the alignment mark 26-3 is patterned on the glass substrate 23-3. In the lamination process, alignment is performed using an alignment mark patterned on each glass substrate.

With this process, produced is the laminated lens wafer 40 in which the resin lenses 24-1 and 24-2 as two layers are laminated. Note that FIG. 6C illustrates one example of the laminated lens wafer 40, and the structure of the laminated lens wafer 40 can be modified in various manners. For example, the number of laminated lenses may be changed. While the above-description has been given of a hybrid lens including the resin lenses 24-1 and 24-2 and the glass substrates 23-1 to 23-3 as an example, the laminated lens wafer 40 is not limited thereto. Alternatively, one layer of a lens may be included in the second optical member 20.

Subsequently, the protrusion 22 serving as the structure for alignment is formed. As illustrated in FIG. 7A, the protrusion 22 is formed on the flat surface 21, which is, out of surfaces of the glass substrate 23-1, a surface on the opposite side of a surface on which the resin lens 24-1 is formed. As described above, in production of the laminated lens wafer 40, alignment is performed using the alignment mark 26 (26-1 to 26-3) as a reference, and the optical axis of the laminated lens wafer 40 is aligned with respect to the alignment mark 26 with high accuracy. In addition, a positional relationship between the recessed portion 13 arranged in the glass lens 30 and the optical axis of the glass lens 30 has been known. Thus, the protrusion 22 can be arranged at a position corresponding to the recessed portion 13 in the glass lens 30 using the alignment mark 26 as a reference. For example, the protrusion 22 is formed in a concentric region with respect to the optical axis of the laminated lens wafer 40, as illustrated in FIG. 7B.

The protrusion 22 is, for example, a photosensitive adhesive. The photosensitive adhesive is a resin that can be dissolved by being exposed to light, and that can be bonded by heating. Alternatively, the protrusion 22 may be a resin that is cured by exposure to ultraviolet light. Photolithography that is widely used in a semiconductor manufacturing process can be applied to formation of the protrusion 22.

2.3 Alignment and Singulation

Alignment, bonding, and singulation processes described in steps S13 to S15 are now described. As described above, the glass lens 30 in which the recessed portion 13, which is the concentric groove about the optical axis, has been produced in step S11. In a case where the recessed portion 13 is a groove that is digged in a region between a circle with a radius r1 and a circle with a radius r2 (>r1), r1 and r2 are known design values. The laminated lens wafer 40 in which the protrusion 22 is formed at the position corresponding to the recessed portion 13 has been produced in step S12. The position corresponding to the recessed portion 13 represents part or the whole of the region between the circle with the radius r1 and the circle with the radius r2 centering on the optical axis of the laminated lens wafer 40 in the plan view. In the example illustrated in FIG. 7B, the protrusions 22 are arranged in the region in an annularly consecutive manner.

The glass lens 30 may be arranged in the laminated lens wafer 40 using a flip-chip bonder or a robot hand. In addition, the arrangement of the glass lens 30 is not prevented from being manually performed.

As illustrated in FIG. 8A, the protrusion 22 of the laminated lens wafer 40 is fitted with the recessed portion 13 of the glass lens 30, whereby alignment between the optical axis of the glass lens 30 and the optical axis of the laminated lens wafer 40 is performed with high accuracy. When an attention is paid to the protrusion 22 at a given position, at least one of contact between a surface of the protrusion 22 on a D1 side and a surface of the recessed portion 13 on the D1 side or contact between a surface of the protrusion 22 on a D2 side and a surface of the recessed portion 13 on the D2 side is achieved. D1 is a direction from the optical axis toward the protrusion 22 and an extended direction, and D2 is an opposite direction thereof. In other words, D1 is an eccentric direction about the optical axis, and D2 is an opposite direction thereof. That is, the recessed portion 13 and the protrusion 22 restrict a relative position between the glass lens 30 and the laminated lens wafer 40 from being deviated in the direction of D1 or the direction of D2. In a case where the protrusion 22 is arranged in the concentric pattern, since a relative position in various directions crossing the optical axis is restricted from being deviated, it is possible to perform alignment between the glass lens 30 and the laminated lens wafer 40 in the x- and y-directions with high accuracy.

In a case where the protrusion 22 is a photosensitive adhesive, the glass lens 30 and the laminated lens wafer 40 are bonded to each other by execution of a curing process such as heating and exposure to light after the alignment.

Subsequently, singulation is performed as illustrated in FIG. 8B. For example, a dicing blade 55 is used for the singulation. However, the singulation is not limited to that using the dicing blade 55, and another method such as laser dicing may be used. A plurality of optical laminates 1 is obtained by the singulation.

As described above, the protrusion 22 in accordance with the present embodiment may be the photosensitive adhesive. The protrusion 22 is then arranged inside the recessed portion 13 in the first optical member 10.

This allows the protrusion 22 serving as the structure for alignment to be used as an adhesive. In the method in accordance with the present embodiment, since the first optical member 10 and the second optical member 20 are fixed in a state where the surface of the substrate 11 and the flat surface 21 as the planes are pushed against each other, it is possible to increase accuracy in alignment in the laminating direction. Especially, in a case where the protrusion 22 arranged inside the recessed portion 13 is used as the adhesive, there is no need for arranging an adhesive between the surface of the substrate 11 on the first optical member 10 side and the flat surface 21 on the second optical member 20 side. This prevents an error due to a thickness of the adhesive, and can thereby further increase accuracy in alignment in the laminating direction.

In addition, since the recessed portion 13 and the protrusion 22 are bonded to each other on the curved surfaces, it is possible to secure bonding strength even if a bonding area in the plan view is smaller than that in a case of bonding between planes. The bonding area in the plan view is an area of a region in which the protrusion 22 is formed when viewed in the laminating direction, and corresponds to an area of the concentric circle in the example illustrated in FIG. 7B.

In addition, the second optical member 20 may be a lens unit in which a plurality of lenses is laminated. In the example illustrated in FIG. 1, the two resin lenses 24-1 and 24-2 are laminated in the second optical member 20. However, the number of laminated lenses may be three or more.

In the method in accordance with the present embodiment, the first optical member 10 is not prevented from being a lens unit in which a plurality of lenses is laminated. For example, the optical laminate 1 may be produced by lamination of two or more layers of the glass lenses 30 in the laminated lens wafer 40. However, when two or more layers of the glass lenses 30 are laminated, the process of aligning the singulated glass lens 30 with high accuracy needs to be executed for the number of layers. That is, the glass lens 30 is more advantageous in optical performance such as a refractive index than the resin lens 24, but there is a possibility that laminating multitudes of glass lenses 30 decreases productivity. In this regard, if optical performance is enhanced by usage of the second optical member 20 as the laminated lens wafer 40, it is possible to pursuit both of productivity and optical performance of the optical laminate 1 simultaneously.

Alternatively, the first optical member may be a glass lens. This enables addition of a lens that has relatively high optical performance to the second optical member 20, and thereby enables enhancement of optical performance, downsizing of the optical laminate 1, and the like. In addition, since the first optical member 10 is a member that is less susceptible to deformation than the protrusion 22, it is possible to prevent distortion of a lens due to alignment.

The recessed portion 13 may be formed in a concentric pattern with respect to the optical axis of the optical window 12. Specifically, the recessed portion 13 is formed in the concentric pattern with respect to the optical axis in a plan view when the first optical member 10 is viewed from a point of view set to the first optical member 10 in a −z-direction. As described above with reference to FIG. 3, the first optical member 10 is subjected to polishing in a rotating state. Alternatively, the first optical member 10 is, when being molded using the metal mold, subjected to polishing in a state where the metal mold is rotating. If the glass plate 31 or the metal mold is removed from the rotating table 51 during polishing, the rotating center of subsequent polishing is deviated. Thus, it becomes difficult to form a pattern with the optical axis serving as a reference. In other words, forming the recessed portion 13 in the concentric pattern with respect to the optical axis of the optical window 12 enables arrangement of the structure for alignment in which the positional relationship with the optical axis is defined with high accuracy.

In addition, as illustrated in FIG. 7B, the protrusion 22 may be formed in a concentric pattern in a region corresponding to the recessed portion 13 formed in a concentric pattern. Arranging the protrusion 22 having a continuous circular shape in this manner restricts positional deviation in a freely-selected direction on a plane crossing the laminating direction. As a result, it becomes possible to perform alignment between the first optical member 10 and the second optical member 20 using the recessed portions 13 and the protrusions 22 with high accuracy.

Additionally, the first optical member may include a glass substrate. The glass substrate mentioned herein is the glass substrate 23-1 in a more limited sense. The flat surface 21 of the second optical member 20 is a surface of the glass substrate 23-1 on the first optical member 10 side. This allows the flat surface 21 of the second optical member 20 to be a glass surface. In the method in accordance with the present embodiment, since the flat surface of the substrate 11 of the first optical member 10 and the flat surface 21 of the second optical member 20 are fixed in a state where the planes are in contact with other, it is possible to increase accuracy in alignment in the laminating direction. At this time, forming the plane using glass, which is a material that is less susceptible to deformation than a resin or the like, can further increase accuracy in alignment in the laminating direction. However, the flat surface 21 of the second optical member 20 is not prevented from being a surface made of a member other than glass such as a plane of a silicon substrate.

The optical laminate 1 in accordance with the present embodiment includes: the first optical member 10 where the optical window 12 is formed in the planar substrate 11, the first optical member 10 including the recessed portion 13 around the optical window 12; and the second optical member that has the flat surface 21 facing the substrate 11 of the first optical member 10, and that includes, on the flat surface 21, the protrusion 22 made of resin for being fitted with the recessed portion 13. A Young's modulus of the protrusion 22 is smaller than a Young's modulus of the first optical member 10. The Young's modulus mentioned herein is a coefficient representing a relationship between stress applied to an object in a given direction and an amount of distortion in the direction. As the Young's modulus becomes smaller, the object is more likely to be distorted in response to stress. That is, the protrusion 22 in accordance with the present embodiment is a member that is more likely to be distorted than the first optical member 10.

As described above, in the method in accordance with the present embodiment, arranging the protrusion 22 on the flat surface 21 can prevent a decrease in optical performance of the second optical member 20. Meanwhile, the recessed portion 13 serving as the structure for alignment of the first optical member 10 is arranged integrally with the substrate 11 in which the optical window 12 is formed. Hence, to prevent a decrease in optical performance of the first optical member 10, members of the first optical member 10 and the protrusion 22 need to be taken into consideration. In this respect, in a case where the Young's modulus of the protrusion 22 is smaller than the Young's modulus of the first optical member 10, the protrusion 22 is relatively more susceptible to deformation, so that distortion of the first optical member 10 can be prevented.

The method in accordance with the present embodiment can be applied to a manufacturing method for the optical laminate 1 illustrated in FIG. 3. The manufacturing method for the optical laminate 1 includes a process of producing the glass lens 30, a process of producing the lens unit, a process of forming the protrusion 22, a process of attaching a plurality of glass lenses 30 to the lens unit, and a process of performing singulation into a plurality of optical laminates 1.

The process of producing the glass lens 30 is performed by formation of the optical window 12 in the planar glass plate 31 and formation of the recessed portion 13 or the protruding portion 14, which will be described later, around the optical window 12 in the glass plate 31, as illustrated in FIGS. 4 and 5. The lens unit mentioned herein includes one or more lens wafers, and has the flat surface 21. As illustrated in FIG. 6C, in a case where a plurality of lens wafers is laminated, the lens unit is the laminated lens wafer 40 (wafer level lens before singulation). The protrusion 22 is made of resin, and is arranged on the flat surface 21 of the lens unit, as illustrated in FIGS. 7A and 7B. The process of attaching the plurality of glass lenses 30 to the lens unit is performed by fitting of the recessed portion 13 or protruding portion 14 of the glass lens 30 with the protrusion 22 of the lens unit, as illustrated in FIG. 8A. The protruding portion 14 will be described later. The process of performing singulation into the optical laminate 1 is performed by, for example, dicing using the dicing blade 55, as illustrated in FIG. 8B. The method in accordance with the present embodiment can implement the manufacturing method for the optical laminate 1 that is well balanced between productivity and optical performance.

### 3. Other Configuration Examples of Optical Laminate

Some modifications will be described below. Note that the following description will be given mainly of the optical laminate 1 after singulation. Hence, an example in which the first optical member 10 has a rectangular shape in a plan view is described. As described above, the glass lens 30 has a disk shape, and the first optical member 10 in a rectangular shape may be formed by dicing. Alternatively, the glass lens 30 in a rectangular shape may be produced or bonded. In a case where part of the glass lens 30 is removed by dicing, presence/absence of the recessed portion or the protruding portion in the part to be removed and a width of the recessed portion or the like can be modified in various manners.

The same applies to the second optical member 20, and the description will be given mainly of the portion after singulation among the laminated lens wafer 40. While a remaining portion of the protrusion 22 after dicing is illustrated, the protrusion 22 may be arranged in a range that is removed by dicing. For simplification of the description, one glass substrate 23 arranged on the first optical member 10 side and the resin lens 24, among the second optical member 20, are illustrated, and illustration of the other components is omitted.

3.1 Adhesive

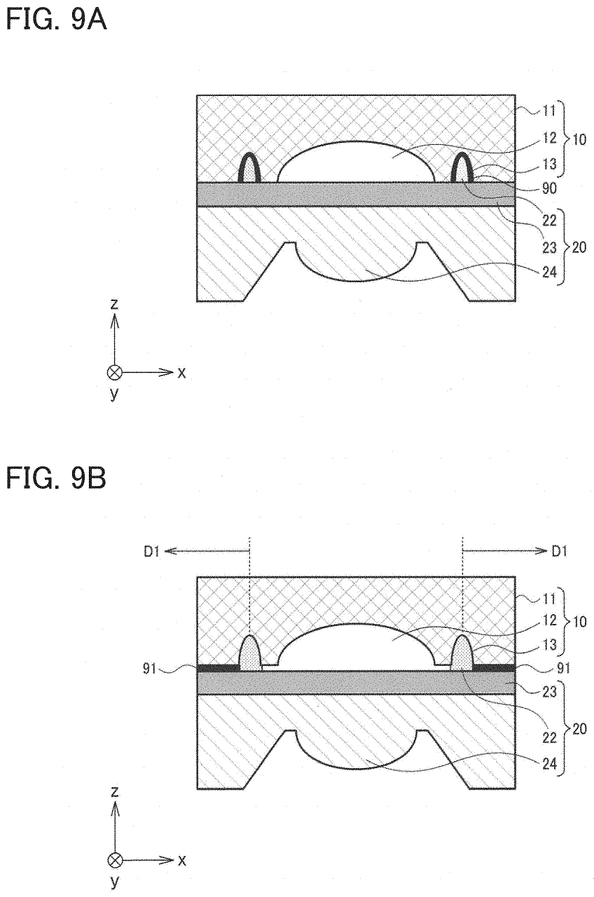

FIGS. 9A and 9B are diagrams for describing a modification regarding bonding. As illustrated in FIG. 9A, an adhesive 90 is arranged on the surface of the protrusion 22, and the protrusion 22 may be arranged inside the recessed portion 13 of the first optical member 10. That is, the protrusion 22 in this case is the structure for alignment, and the protrusion 22 itself does not function as an adhesive. The adhesive 90 is used aside from the protrusion 22. The protrusion 22 may be formed using photolithography similarly to the above-mentioned example, or may be formed using a dispenser to apply resin. Also in this case, since bonding is performed inside the recessed portion 13, it is possible to increase accuracy in alignment in the laminating direction. The adhesive mentioned herein is ultraviolet curable resin, but another adhesive may be used.

As illustrated in FIG. 9B, the first optical member 10 and the second optical member 20 are bonded to each other with an adhesive 91, and the adhesive 91 may be arranged outside the protrusion 22 with respect to the optical axis of the optical laminate 1. Specifically, among the surface of the substrate 11 constituting the first optical member 10 on the second optical member 20 side, a flat portion representing a region excluding the optical window 12 and the recessed portion 13 and the flat surface 21 of the second optical member 20 are bonded to each other. Being outside the protrusion 22 with respect to the optical axis represents a position that is more advanced in the direction of D1 than the protrusion 22. D1 is a direction from the optical axis toward the protrusion 22, and is a direction orthogonal to the optical axis, in a more limited sense.

In this case, the adhesive 91 is arranged between the flat surface of the substrate 11 of the first optical member 10 and the flat surface 21 of the second optical member 20. Thus, to increase accuracy in alignment in the laminating direction, a thickness of the adhesive 91 be adjusted. In the configuration in FIG. 9B, a bonded portion is not limited to the inside of the recessed portion 13. Thus, the configuration is advantageous in that a bonding area in a plan view can be increased. Additionally, since the adhesive 91 is arranged outside the protrusion 22, the protrusion 22 can prevent the adhesive 91 from flowing in the optical axis direction. That is, the configuration can prevent a decrease in optical performance due to circulation of the adhesive 91 in an effective range of a lens.

3.2 Structure for Alignment

Subsequently, a modification regarding the structure for alignment arranged in the first optical member 10 and the second optical member 20 is described.

FIG. 10A is a plan view of the first optical member 10. FIG. 10B is a plan view of the second optical member 20. FIG. 10C is a cross-section view of the optical laminate 1. As illustrated in FIGS. 10A to 10C, the recessed portion 13 serving as the structure for alignment may be arranged to be continuous to the recessed portion (optical window 12) serving as lens structure. The protrusion 22 of the second optical member 20 is arranged at a position corresponding to the recessed portion 13.

As illustrated in FIG. 10C, when an attention is paid to the protrusion 22 at a given position, the surface of the protrusion 22 on the D1 side and the surface of the recessed portion 13 on the D1 side are in contact with each other, and the surface of the protrusion 22 on the D2 side is not in contact with the recessed portion 13. Fitting of the recessed portion 13 and the protrusion 22 with each other restricts movement of the first optical member 10 in the direction of D2. In a case where the protrusion 22 is arranged in the concentric pattern, since a relative position in various directions crossing the optical axis is restricted from being deviated, it is possible to perform alignment between the first optical member 10 and the second optical member 20 with high accuracy.

FIG. 11A is a plan view illustrating another configuration of the first optical member 10. FIG. 11B is a plan view of the second optical member 20. FIG. 11C is a cross-section view of the optical laminate 1.

As illustrated in FIGS. 11A and 11C, a first recessed portion 13-1 and a second recessed portion 13-2 are arranged in the surface of the substrate 11 of the first optical member 10, whereby the protruding portion 14 may be formed between the first recessed portion 13-1 and the second recessed portion 13-2. The protrusion 22 is arranged at a position corresponding to at least one of the first recessed portion 13-1 or the second recessed portion 13-2. FIGS. 11B and 11C each illustrate an example in which the protrusion 22 is arranged at a position corresponding to the first recessed portion 13-1. When an attention is paid to the protrusion 22 at a given position, the surface of the protrusion 22 on the D1 side and the surface of the protruding portion 14 on the D2 side are in contact with each other. Fitting of the protruding portion 14 and the protrusion 22 with each other restricts movement of the first optical member 10 in the direction of D2.

As illustrated in FIGS. 11A to 11C, the optical laminate 1 in accordance with the present embodiment includes: the first optical member 10 where the optical window 12 is formed in the planar substrate 11, the first optical member 10 including the protruding portion 14 around the optical window 12; and the second optical member that has the flat surface 21 facing the substrate 11 of the first optical member 10, and that includes, on the flat surface 21, the protrusion 22 made of resin for being fitted with the protruding portion 14. That is, the structure for alignment of the first optical member 10 may be the recessed portion 13 or the protruding portion 14.

Also when the protruding portion 14 is formed on the first optical member 10, the point that the protruding portion 14 is formed in the concentric pattern with respect to the optical axis of the optical window 12 is similar to that in the case of forming the recessed portion 13. This enables arrangement of the protruding portion 14 with respect to the optical axis with high accuracy. The protrusion 22 has, for example, a circular shape corresponding to the protruding portion 14 formed in the concentric pattern.

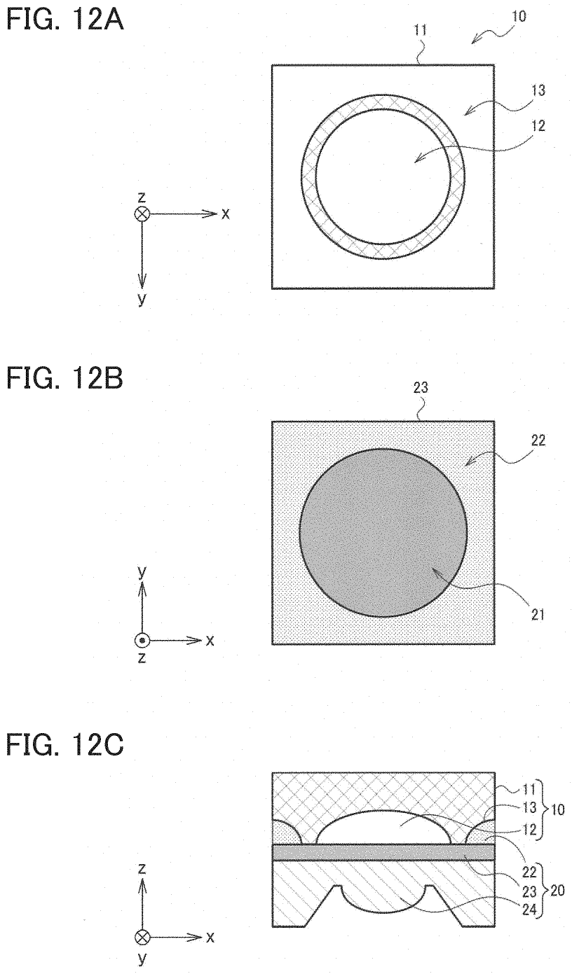

FIG. 12A is a plan view illustrating still another configuration of the first optical member 10. FIG. 12B is a plan view of the second optical member 20. FIG. 12C is a cross-section view of the optical laminate 1.

As illustrated in FIG. 12A, the recessed portion 13 formed in the first optical member 10 may be continuously formed to the outer periphery of the first optical member 10. As illustrated in FIG. 12B, the protrusion 22 is arranged at a position corresponding to the recessed portion 13. The configuration illustrated in FIGS. 12A to 12C can increase a contact area in a plan view in comparison with the configuration illustrated in FIG. 7B or the like.

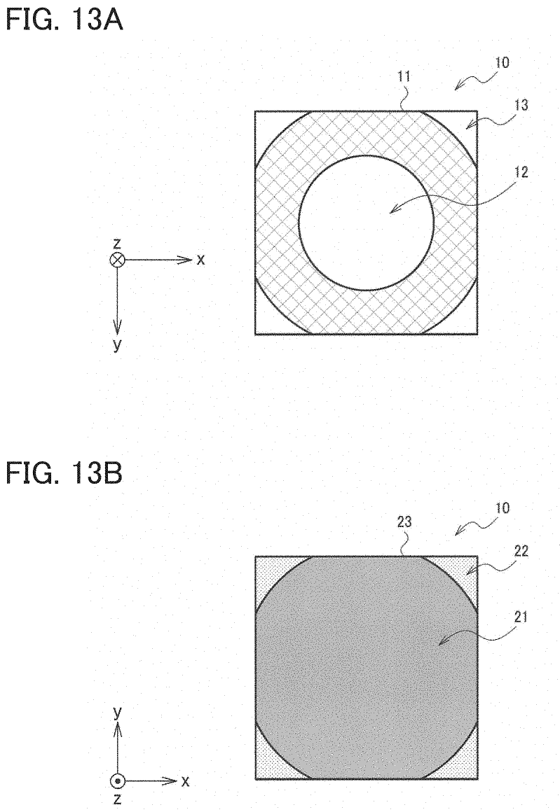

FIG. 13A is a plan view illustrating still another configuration of the first optical member 10. FIG. 13B is a plan view of the second optical member 20. As illustrated in FIGS. 13A and 13B, the recessed portion 13 and the protrusion 22 may be configured to partially remain at four corners of the optical laminate 1. The first optical member 10 and the second optical member 20 are bonded to each other at the four corners of the optical laminate 1.

As described above, the recessed portion 13 or the protruding portion 14 serving as the structure for alignment of the first optical member 10 and the protrusion 22 serving as the structure for alignment of the second optical member 20 are only required to be arranged in a corresponding positional relationship with respect to the optical axis. Additionally, in the method in accordance with the present embodiment, the surface of the protrusion 22 on the D1 side or the surface of the protrusion 22 on the D2 side is only required to be capable of being fitted with the structure for alignment of the first optical member 10, and a specific shape of the recessed portion 13 or the protruding portion 14 and a specific shape of the protrusion 22 can be modified in various manners.

3.3 Light Shielding Member

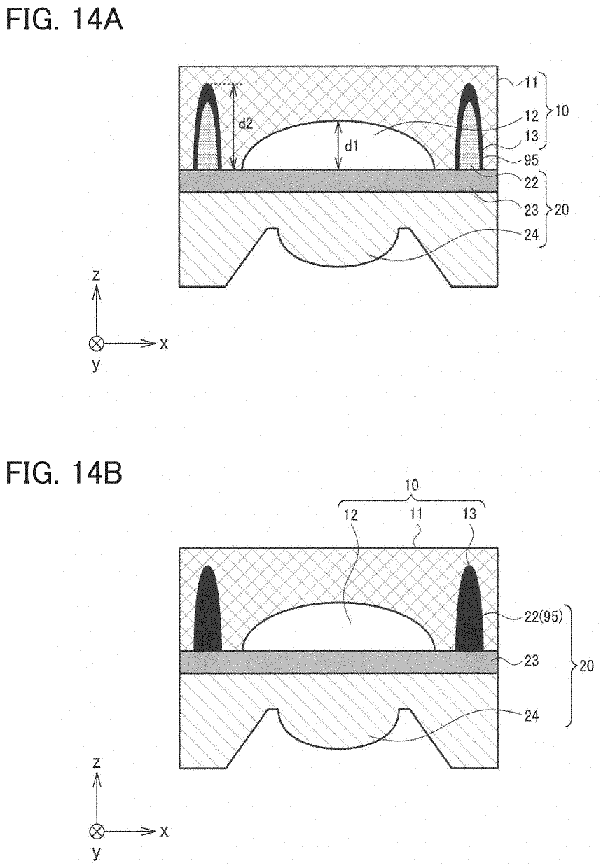

FIGS. 14A and 14B are cross-section views each illustrating another configuration of the optical laminate 1. As illustrated in FIGS. 14A and 14B, light shielding resin 95 may be filled inside the recessed portion 13. At this time, in the laminating direction of the first optical member 10 and the second optical member 20, a depth d2 of the recessed portion 13 with respect to the flat surface of the substrate 11 of the first optical member 10 is greater than a depth d1 of the optical window 12 with respect to the surface of the substrate 11. The depth d1 of the optical window 12 mentioned herein represents a distance in the deepest portion of the substrate 11 in a thickness direction with the surface of the substrate 11 on the second optical member 20 side serving as a reference. In a case where the optical window 12 is a concave lens illustrated in FIGS. 14A and 14B, the depth d1 corresponds to a distance at a position corresponding to the optical axis AX. Alternatively, the optical window 12 may be a convex lens. The depth d1 in this case corresponds to a distance in a peripheral portion of an effective range of the convex lens. That is, the distance d1 mentioned herein is only required to be a distance of the deepest portion among the optical window 12, and a specific position on the x-y plane can be modified in various manners.

This can prevent incident of disturbance light on the optical window 12 of the first optical member 10. Note that a light shielding member can be arranged on a side surface of the optical laminate 1 to prevent incidence of disturbance light. In a conventional method of laminating glass lenses, a lens barrel serves as a light shielding member, and incidence of light from the side surface is prevented. However, since the optical laminate 1 in accordance with the present embodiment is subjected to the singulation process such as dicing, a cross-section shape is assumed to be a rectangular shape. Hence, in comparison with a lens unit using a glass lens whose cross-section shape is a circular shape and a lens barrel, a distance from the side surface to the optical window 12 on a diagonal line of the optical laminate 1 becomes larger. As a result, even with arrangement of the light shielding member on the side surface, there is a possibility that light that has not appropriately passed through the optical window 12 enters the inside of the optical laminate 1. In this respect, filling the light shielding resin 95 inside the recessed portion 13 enables blocking of disturbance light at a position closer to the optical window 12 than the side surface to the optical window 12. At this time, making the recessed portion 13 deeper can enhance light shielding performance.

Note that the light shielding resin 95 may be filled between the protrusion 22 and the recessed portion 13 as illustrated in FIG. 14A. Alternatively, as illustrated in FIG. 14A, the protrusion 22 may serve as the light shielding resin 95. That is, the light shielding member may be arranged aside from the protrusion 22 serving as the structure for alignment, or the protrusion 22 serving as the structure for alignment may serve as the light shielding member. A specific structure can be modified in various manners.

3.4 Shape of Protrusion

The description has been given of the example in which the protrusion 22 having the continuous circular shape is arranged. For example, the protrusion 22 is arranged in a concentric pattern corresponding to the recessed portion 13. As described above, the recessed portion 13 or the protruding portion 14 is formed while the glass plate 31 is rotated using the rotating table 51 (refer to FIG. 4), and it is not easy to have a configuration other than the concentric pattern. However, the protrusion 22 is only required to be capable of being fitted with the recessed portion 13 or the protruding portion 14, and the specific shape can be modified in various manners.

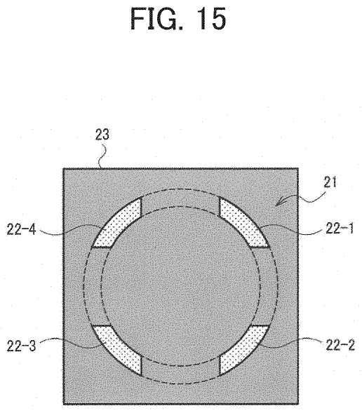

As illustrated in FIG. 15, for example, the protrusion 22 may be arranged in a partial region among a concentric pattern region corresponding to the recessed portion 13 or the protruding portion 14 formed in the concentric pattern. FIG. 15 illustrates an example in which four protrusions 22-1 to 22-4 are arranged. Note that a size of each protrusion 22 is not limited to that illustrated in FIG. 15, and can be modified in various manners. For example, in a plan view when observed from the Z-direction, a plurality of dot-like protrusions 22 may be arranged. Arranging three or more dot-like protrusions 22 can restrict relative movement of the first optical member 10 in the x-y direction. In addition, sizes of the plurality of protrusions 22 are not necessarily matched, and a plurality of protrusions 22 having different sizes may be arranged.

The number of protrusions 22 is not limited to a plural number, and one protrusion 22 may be arranged. When one protrusion 22 is arranged and is small in size, the protrusion 22 can restrict movement of the first optical member 10 in at least one of a direction from the protrusion 22 toward the optical axis, or the opposite direction thereof, but it is difficult for the protrusion 22 to restrict movement of the first optical member 10 in other directions. However, since it is possible to perform alignment in at least one direction, the configuration can increase alignment accuracy in comparison with a case where no protrusion 22 is arranged. In a case where alignment accuracy needs to be further increased, it is preferable that two or more protrusions 22 be arranged, or the protrusion 22 have a larger size.

### 4. Endoscope System

The method in accordance with the present embodiment can be applied to an endoscope 2. The endoscope 2 mentioned herein is, specifically, an endoscopic scope, and includes the insertion section 100. The insertion section 100 includes the optical laminate 1 on which object light representing light from the object is incident, and an image sensor 5 that captures an image based on the object light that has passed through the optical laminate 1. As described above, the optical laminate 1 includes: the first optical member 10 where the optical window 12 is formed in the planar substrate 11, the first optical member 10 including the recessed portion 13 or the protruding portion 14 around the optical window 12; and the second optical member that has the flat surface 21 facing the substrate 11 of the first optical member 10, and that includes, on the flat surface 21, the protrusion 22 made of resin for being fitted with the recessed portion 13 or the protruding portion 14.

FIG. 16A illustrates a configuration example of the endoscope system 3 including the endoscope 2. As illustrated in FIG. 16A, the endoscope system 3 includes the endoscope 2 in accordance with the present embodiment, a processor 300, and a monitor 400. The insertion section 100 having a long and thin shape is inserted to a body cavity of a subject, and the endoscope 2 thereby captures an image of the inside of the body of the subject and outputs a captured image signal.

The endoscope 2 includes the insertion section 100, a grasping section 200 that is arranged on a base end section side of the insertion section 100, a universal code 220 that is extended from the grasping section 200, and a connector 230 that is arranged on the base end section side of the universal code 220. The insertion section 100 includes a rigid leading end section 110 in which an imaging module 111 is arranged, a bending section 120 that is extended on the base end side of the leading end section 110, that can be bended freely, and that is used for changing a direction of the leading end section 110, and a flexible section 130 that is extended on the base end side of the bending section 120. The endoscope 2 is a flexible scope, but may be a rigid scope. That is, the flexible section or the like is not an essential constituent element. An angle knob 210 serving as an operating section that is turned and that is used for an operator to operate the bending section 120 is arranged in the grasping section 200.

FIG. 16B is a cross-section view illustrating a configuration example of the imaging module 111. As illustrated in FIG. 16B, the imaging module 111 arranged in the leading end section 110 includes the optical laminate 1 and the image sensor 5. On a surface of the image sensor 5 on the opposite side of the optical laminate 1, a cable section 140 is arranged so as to be in contact with the surface. Alternatively, on the surface of the image sensor 5 on the opposite side of the optical laminate 1, a peripheral circuit chip, which is not illustrated, is arranged, and the cable section 140 may be connected to the peripheral circuit chip. A peripheral circuit for controlling and driving the image sensor 5 is arranged on the peripheral circuit chip.

In the example illustrated in FIG. 16B, the cable section 140 includes a signal cable 141, and a flexible print substrate (flexible printed circuit (FPC) substrate 142). The signal cable 141 is connected to the image sensor 5 via the FPC substrate 142. The signal cable 141 is extended to the connector 230 via the universal code 220. Although not illustrated in FIGS. 16A and 16B, the endoscope system 3 may include a light source device, a light guide cable that guides illumination light from the light source device to the leading end section 110, and an illumination lens that emits the illumination light toward an object. The optical laminate 1 receives light from the object and forms an image as an object image. The light from the object is, specifically, reflected light that is illumination light reflected on the object.

The image sensor 5 may be a charge-coupled device (CCD) image sensor, a complementary metal-oxide semiconductor (CMOS) image sensor, or an element of another system. In addition, the image sensor 5 may be a monochrome sensor, or may be an element having a color filter. The color filter may be a color filter in a well-known Bayer's arrangement, a complementary color filter, or another color filter. The complementary filter includes filters in respective colors of cyan, magenta, and yellow.

The universal code 220 is connected to the processor 300 via the connector 230. The processor 300 controls the whole of the endoscope system 3, and also performs signal processing on a captured image signal output from the imaging module 111 to output the signal as an image signal. The monitor 400 displays the image signal output from the processor 300 as an endoscope image.

In accordance with the present embodiment, the optical laminate 1 described above can be applied to the imaging module 111 of the endoscope 2. This can prevent distortion of an optical member included in the optical laminate 1, and can thereby present an image with high viewability. The use of the optical laminate 1 eliminates the need for a frame body such as a lens barrel, and can thereby make the insertion section 100 thinner Therefore, it is possible to decrease invasiveness in diagnosis and treatment using the endoscope 2.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An endoscope comprising:
an optical laminate on which object light is incident, the object light being light from an object; and
an image sensor that captures an image based on the object light that has passed through the optical laminate, wherein
the optical laminate includes:
a first optical member where an optical window is formed in a planar substrate, the first optical member including a recessed portion or a protruding portion around the optical window; and
a second optical member that has a flat surface facing the substrate of the first optical member, and that includes, on the flat surface, a protrusion made of resin for being fitted with the recessed portion or the protruding portion,
the first optical member is a glass lens,
the second optical member includes a resin lens and a glass substrate,
the flat surface of the second optical member is a surface of the glass substrate on the first optical member side,
the resin lens is arranged on, out of surfaces of the glass substrate, a surface on an opposite side of the flat surface, and is not arranged on the flat surface, and
a flat portion excluding the optical window and the recessed portion or the protruding portion among the substrate of the first optical member is in contact with the flat surface that is a substrate surface of the glass substrate of the second optical member.

2. An optical laminate comprising:
a first optical member where an optical window is formed in a planar substrate, the first optical member including a recessed portion or a protruding portion around the optical window; and
a second optical member that has a flat surface facing the substrate of the first optical member, and that includes, on the flat surface, a protrusion made of resin for being fitted with the recessed portion or the protruding portion, wherein
the first optical member is a glass lens,
the second optical member includes a resin lens and a glass substrate,
the flat surface of the second optical member is a surface of the glass substrate on the first optical member side,
the resin lens is arranged on, out of surfaces of the glass substrate, a surface on an opposite side of the flat surface, and is not arranged on the flat surface, and
a flat portion excluding the optical window and the recessed portion or the protruding portion among the substrate of the first optical member is in contact with the flat surface that is a substrate surface of the glass substrate of the second optical member.

3. The optical laminate as defined in claim 2, wherein
the protrusion is a photosensitive adhesive, and
the protrusion is arranged inside the recessed portion of the first optical member.

4. The optical laminate as defined in claim 2, wherein
an adhesive is arranged on a surface of the protrusion, and
the protrusion is arranged inside the recessed portion of the first optical member.

5. The optical laminate as defined in claim 2, wherein
the first optical member and the second optical member are bonded to each other with an adhesive, and
the adhesive is arranged outside the protrusion with respect to an optical axis of the optical laminate.

6. The optical laminate as defined in claim 2, wherein
in a laminating direction of the first optical member and the second optical member, a depth of the recessed portion with respect to the flat surface of the substrate of the first optical member is greater than a depth of the optical window, and
light shielding resin is filled inside the recessed portion.

7. The optical laminate as defined in claim 6, wherein the light shielding resin is filled between the protrusion and the recessed portion.

8. The optical laminate as defined in claim 6, wherein the protrusion is made of the light shielding resin.

9. The optical laminate as defined in claim 2, wherein the second optical member is a lens unit in which a plurality of lenses is laminated.

10. The optical laminate as defined in claim 2, wherein the recessed portions or the protruding portion is formed in a concentric pattern with respect to an optical axis of the optical window.

11. The optical laminate as defined in claim 10, wherein the protrusion is formed in a concentric pattern in a region corresponding to the recessed portion or the protruding portion.

12. The optical laminate as defined in claim 10, wherein the protrusion is arranged in a partial region of a concentric region corresponding to the recessed portion or the protruding portion.

13. A manufacturing method for an optical laminate, comprising:
producing a glass lens by forming an optical window in a planar glass plate and forming a recessed portion or a protruding portion around the optical window of the planar glass plate;
producing a lens unit including one or more lens wafers and having a flat surface;
forming, on the flat surface of the lens unit, a protrusion made of resin;
attaching a plurality of glass lenses to the lens unit by fitting the recessed portion or protruding portion of the glass lens with the protrusion of the lens unit; and
performing singulation into a plurality of optical laminates by cutting a wafer, wherein
the producing the glass lens includes execution of producing of the glass lens that has been subjected to singulation multiple times to produce a plurality of glass lenses,
the attaching the plurality of glass lenses to the lens unit includes attaching the plurality of glass lenses to the lens unit by fitting the recessed portion or protruding portion of the glass lens with the protrusion of the lens unit and bringing a flat portion of the glass plate and the flat surface of the lens unit into contact with each other,
the lens unit includes a resin lens and a glass substrate,
the flat surface of the lens unit is a surface of the glass substrate on the glass lens side, and
the producing the lens unit includes arranging the resin lens on an opposite side of the flat surface without arranging the resin lens on the flat surface.
